(19) United States
(12) Patent Application Publication
(10) Pub. No.: US 2018/0190369 A1
(43) Pub. Date: Jul. 5, 2018

(54) METHODS, SYSTEMS AND TOOLS FOR SELECTING SUBJECTS SUFFERING FROM NEURODEGENERATIVE DISEASE

(71) Applicant: IXICO Technologies Limited, London (GB)

(72) Inventors: Robin Mitja Benjamin Wolz, London (GB); Derek Lionel Glendon Hill, London (GB)

(21) Appl. No.: 15/738,864

(22) PCT Filed: Jun. 29, 2016
(86) PCT No.: PCT/GB2016/051940

§ 371 Date: Dec. 21, 2017

(30) Foreign Application Priority Data
Jun. 29, 2015 (GB) 1511372.3

(51) Int. Cl. G16H 10/20; G16H 50/70

(52) U.S. Cl. CPC G16H 10/20; G16H 50/30; G16H 50/70

(57) ABSTRACT

A system for characterizing a patient as being suitable or non-suitable for treatment of a neurodegenerative disease, the system comprising: a memory for storing data relating to patients, wherein the data includes a plurality of patient specific data types from a first data set and a plurality of patient specific data types from a second data set, different to the first data set; a first data input source for inputting patient specific data into the memory from the first data set and a second data input source for inputting patient specific data into the memory from the second data set; a processor for manipulating and/or combining patient specific data stored in the memory from the first data set and data stored in the memory from the second data set to define an enrichment indicator and compare said enrichment indicator to a pre-determined target indicator and an output for displaying patients whom display enrichment indicators that correlate to the pre-determined target indicator.

The present invention relates to methods, systems and tools for selecting individuals who suffer from neurodegenerative diseases for treatment.

### BACKGROUND

Following disappointing results in recent Phase II/III trials in MCI and mild AD populations, more detailed patient stratification protocols play an increasingly important role in trial design. Patient demographics, clinical scores on cognition and function as well as different biomarkers have all been used for defining specific inclusion criteria. Different work was proposed recently for patient stratification (predominantly in MCI populations) that look at individual markers or multi-stage approaches where multiple markers are used sequentially. As there is an interplay between the different measurements, a combinatorial approach that considers such relationships is hypothesized to be more powerful than a purely sequential approach where every marker is considered independently.

When selecting individuals for treatment in a clinical trial or in the clinic, selection is traditionally based on measurable clinical criteria. When treating neurodegenerative diseases, clinical criteria typically require patients to exhibit impairments in both cognitive and functional domains. Clinical trials in relation to Alzheimer's disease, for example, are thus restricted to patients in the later phases of Alzheimer's disease where patients display measurable symptoms. However, it is well recognized that in the very early stages of Alzheimer's disease, sometimes referred to as “pre-clinical”, patients will undergo underlying anatomical and pathophysiologic changes which occur several years prior to patients exhibiting measurable clinical symptoms. In this pre-clinical disease setting, the traditional clinical approach is not adequate. To identify which of those pre-symptomatic patients are appropriate for treatment in clinical practice or clinical trials requires the analysis of multiple sources of data and the combination of that data to identify patients with the appropriate phenotype.

The data to be combined may include measurements of patient demographics, clinical scales, biomarkers (imaging, molecular etc), multi-domain cognitive tests, and biosensors. The cognitive scales might include 100s of individual measurements, and the imaging assessments may include 1000s or millions, and biosensor data might include 10s of millions of measurements.

The present invention seeks to remove current limitations on selecting individuals that suffer from neurodegenerative disease for both clinical trials of experimental treatment, and for prescribing approved treatments by providing an integrated assessment of multi-dimensional patient data as opposed to a sequential assessment of individual measurements that does not take into consideration dependencies between individual measurements.

### SUMMARY OF THE INVENTION

An aspect of the invention provides a system for selecting a patient for treatment, the system comprising: i) a memory for storing data relating to patients, wherein the data includes a plurality of patient specific data types from a first data set and a plurality of patient specific data types from a second data set, different to the first data set; ii) a first data input source for inputting patient specific data into the memory from the first data set and a second data input source for inputting patient specific data into the memory from the second data set; iii) a processor for manipulating and/or combining patient specific data stored in the memory from the first data set and data stored in the memory from the second data set to define an enrichment indicator and comparing said enrichment indicator to a pre-determined target indicator, wherein, each patient's enrichment indicator defines a unique variable and the pre-determined target indicator applies a threshold to the enrichment indicator such that patients whose unique variable corresponds to the pre-determined target indicator are selected; and iv) an output for displaying selected patients.

Another aspect of the invention provides a method of selecting a patient for treatment, the method comprising the steps of: (a) collecting a first sub-set of patient specific data comprising at least two of: i) demographic information; ii) medical history; iii) clinical symptoms; iv) subjective complaints and v) activity from a wearable sensor (b) collecting a second sub-set of patient specific data comprising at least two of: vi) clinical test results; vii) diagnostic imaging; viii) CSF analysis; ix) blood based markers and x) genetic risk factors; (c) combining the first sub-set of patient specific data and the second sub-set of patient specific data to define an enrichment indicator; (d) comparing the enriched indicator with a set of pre-determined target indicators; (e) characterizing one or more patients from which the first and second sub-sets of patient specific data were derived as being suitable or non-suitable for treatment of a neurodegenerative disease in accordance with step (d); and (f) selecting one or more patients for treatment.

The invention seeks to improve the quality of assigning patients to treatment relating to certain neurodegenerative diseases, both in clinical trials and healthcare. Taking Alzheimer's disease as an example, it is recognized that certain biomarkers indicate pathophysiologic changes in subjects suffering from the very early stages of Alzheimer's disease several years before clinical impairment is observable. It is desirable to perform clinical trials on drugs and methods of treatment for Alzheimer's diseases on subjects whom are suffering from various stages of the disease. This is not currently possible due to the limitations described above. Once treatment is approved for a certain patient population, appropriate selection criteria need to be applied in the clinic.

Another aspect of the invention provides method of selecting a patient for treatment, the method comprising the steps of: (a) analyzing a patient derived sample for the presence of the markers of Table 2, and (b) characterizing the patient from which the sample was derived as being suitable or non-suitable for treatment, based on the results of step (a), wherein, the characterization step (b) is performed by reference or comparison to two or more of i) demographic information; ii) medical history; iii) clinical symptoms; iv) subjective complaints and v) activity.

Another aspect of the invention provides method of selecting a patient for treatment, the method comprising the steps of: (a) collecting a first sub-set of patient specific data comprising at least two of: i) demographic information; ii) medical history; iii) clinical symptoms; iv)subjective complaints and v) activity from a wearable sensor; (b) collecting a second sub-set of patient specific data comprising predicted progression of a neurodegenerative disease and/or predicted response to treatment of a neurodegenerative disease; (c) combining the first sub-set of patient specific data and the second sub-set of patient specific data to define an enrichment indicator; (d) comparing the enrichment indicator with a set of pre-determined target indicators; (e) characterizing one or more patients from which the first and second sub-sets of patient specific data were derived as being suitable or non-suitable for treatment of a neurodegenerative disease in accordance with step (d); and (f) selecting one or more patients for initial treatment of a neurodegenerative disease.

Another aspect of the invention provides method of selecting a patient for treatment, the method comprising the steps of: (a) collecting a first sub-set of patient specific data comprising at least two of: i) demographic information; ii) medical history; iii) clinical symptoms; iv)subjective complaints and v) activity; (b) collecting a second sub-set of patient specific data comprising at least two of: vi) clinical test results; vii) imaging; viii) CSF analysis; ix) blood based markers and x) genetic risk factors; (c) combining the first sub-set of patient specific data and the second sub-set of patient specific data to define an enrichment indicator; (d) comparing the enrichment indicator with a set of pre-determined target indicators; (e) characterizing one or more patients from which the first and second sub-sets of patient specific data were derived as being suitable or non-suitable for treatment of a neurodegenerative disease in accordance with step (d); and (f) selecting one or more patients for initial treatment of a neurodegenerative disease based on the predicted progression of a specified neurodegenerative disease and/or predicted response to treatment of a specified neurodegenerative disease.

Another aspect of the invention provides method of measuring treatment efficacy, the method comprising: (a) collecting a first sub-set of patient specific data comprising at least two of: i) demographic information; ii) medical history; iii) clinical symptoms; iv) subjective complaints and v) activity; (b) collecting a second sub-set of patient specific data comprising at least one of: vi) clinical test results; vii) imaging; viii) CSF analysis; ix) blood based markers and x) genetic risk factors; xi) predicted progression of a neurodegenerative disease, and xii) predicted response to treatment of a neurodegenerative disease; (c) combining the first sub-set of patient specific data and the second sub-set of patient specific data to define an enrichment indicator; (d) comparing the enrichment indicator with a disease model generated from measurements and/or information taken from patients who have not been treated for a specified disease; and (e) identifying treatment efficacy of a specific patient and determining whether treatment should continue, be terminated or be amended.

### FIGURES

Certain embodiments of the invention will now be described by way of reference to the following figures:

FIG. 1 shows a diagrammatic example of a disease model according to embodiments of the present invention

FIG. 2 shows how the fit of patient data to a reference model learned from a large dataset can iteratively be refined.

FIGS. 3 and 4 show the dynamic nature of the disease model through the combination of structural imaging with an amyloid marker or genotype.

FIG. 5 shows how the hospital based system and home based system of the invention interact in the patient journey.

FIG. 6 shows how the device updates the dynamic personalized disease model, outputs from which include care and treatment plans and emergency plans that can be shared with emergency services.

FIG. 7 shows a diagrammatical representation of a system according to aspects of the invention.

FIG. 8 shows effect size achieved with different enrichment strategies. 100th percentile refers to no HV enrichment

FIG. 9 shows for the different enrichment scenarios the screen failure rate, number of subjects that need to undergo screening, the sample sizes required to detect a 25% change in volume over two years as well as the cost of the hypothetical two-arm study.

### DESCRIPTION

Data necessary for selecting patients for treatment of specific conditions is often fragmented and stored in several unconnected locations. For example, a patients first contact with a medical professional, upon realizing that they have a health issue, would typically be their general practitioner. The general practitioner would assess a basic sub-group of patient information to determine the next course of action in diagnosing a particular condition.

For example, the general practitioner would consider the patient's demographic, medical history, clinical symptoms and subjective complaints. The general practitioner would make notes and add these to a file kept on that specific patient. The patient's file might be stored electronically or it might be stored in hardcopy format. Either way, the patient's file is stored in such a way that it is not accessible to third parties in accordance with local data protection laws.

If the general practitioner is of the opinion that the patient is suffering from an, as of yet, undiagnosed condition, the general practitioner might suggest that clinical tests or scans are conducted. Many diseases have specific biomarkers, such as those derived from blood tests, that indicate whether a patient is suffering from a particular disease. The results of such clinical tests might be stored on the general practitioner's file on the patient or they might be stored separately by a consultant or specialist.

Once a disease has been diagnosed, and sometimes beforehand, the patient will be referred to a consultant or specialist for treatment. The consultant or specialist will instruct for further tests such as scans, blood testing and gene testing, for example. The results of these tests will be stored by the consultant in a different location to the data held by the general practitioner on the patient.

Fragmentation of the collected data as well as high number of different measurements hinders a holistic assessment of all data to provide an accurate diagnosis and prognosis, and therefore requires a technical solution to the combination of this data.

Embodiments of the present invention seek to combine data collected in multiple locations which might include data held by general practitioners, data held by other medical professional, but may also include data collected by the patient themselves (eg: wearable devices) to provide an enrichment indicator. The present invention provides a system that assesses the combined data in a combinatorial fashion rather than with a sequential approach as typically done in the current diagnostic process. In that context, the system provides a comprehensive assessment of a potential interplay of different measurements in multi-dimensional data that is not possible by assessing individual data sources independently and therefore cannot be achieved by standard clinical reasoning.

The enrichment indicator could take multiple forms. An example is a unique variable defined by the combined analysis of different types of patient specific data where a higher value indicates that a patient is more suitable for treatment than patients whose associated enrichment indicator returns a lower value, for example. Alternatively, the enrichment indicator variable may simply indicate whether a patient is suitable for treatment or is not suitable for treatment. The output associated with the enrichment indicator is determined by applying a threshold to the enrichment indicator. The threshold is determined by a pre-determined target indicator calculated as an average from at five hundred data points.

The enrichment indicator can be derived from at least two different types of patient specific data that are combined to generate a variable. The variable can be further defined by combining additional types of patient specific data. By comparing the generated variable with the threshold determined by the pre-determined target indicator, a medical professional can identify patients who would be suitable for treatment of a specified condition and patients who would not be suitable. Borderline cases may be further assessed offline. The output of the comparison of the predetermined target indicator to the enrichment indicator may be simple yes or no output or it may be a rating system where each patient is assigned a ranking according to their suitability for treatment. The system may output a list of the top x number patients who meet the necessary criteria for treatment.

Outputs from the system may be on a visual display screen or outputs may be automatically emailed or messaged to a specified email address or mobile phone number, for example. The outputs may be stored in the system memory for further interrogation and to fulfill regulatory obligations.

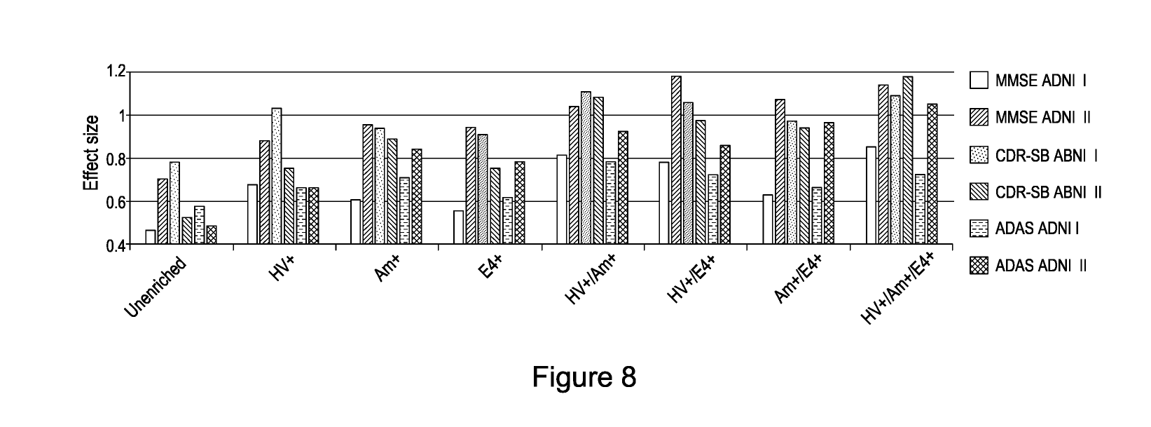

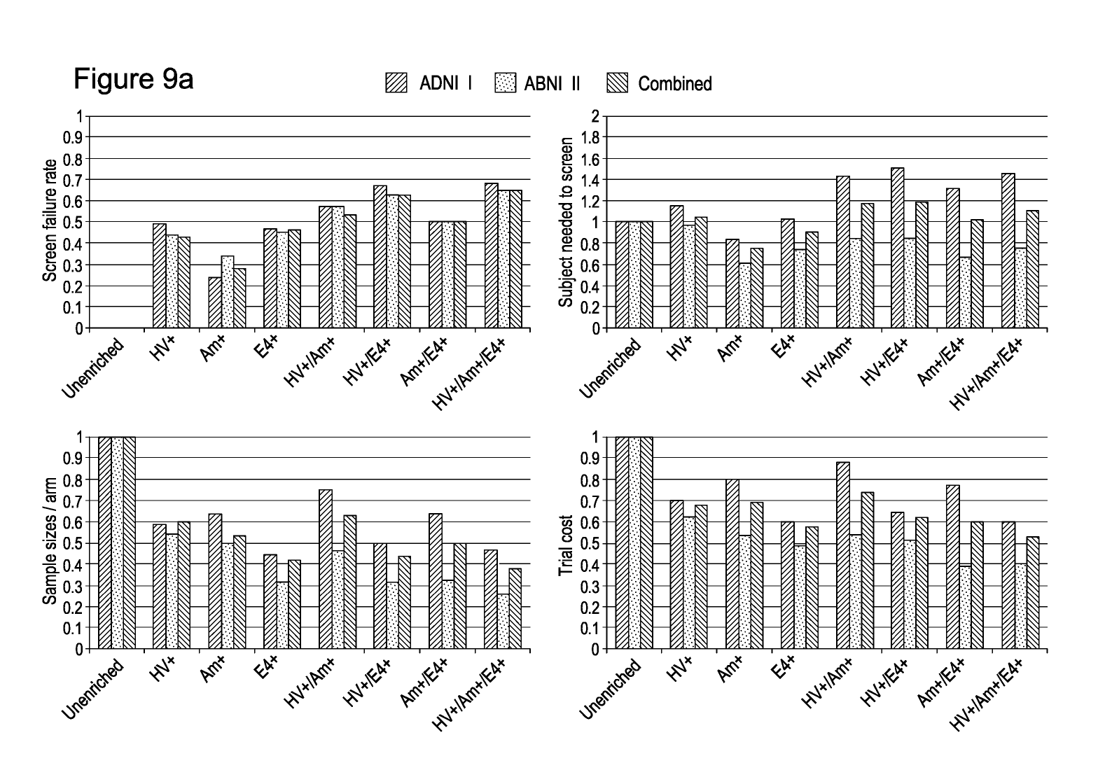

An embodiment of the present invention is illustrated in FIG. 7. The system collects patient-specific data from imaging, clinical tests, activity and demographics. Patient measurements can be collected from a plurality of input sources like general practitioners and hospital sites or through patient self-assessment (e.g. activity measurement, self-reported outcomes). Data is collected from the different measurement locations through a remote data-cloud and stored in a local memory.

For example, basic patient data such as demographic data may be input into the memory from a first location by a general practitioner, for example, and data such as predicted response to treatment of a neurodegenerative disease may be input into the memory from a second location by a consultant or other medical professional. Data may also be collected through wearable sensors. Data including heart rate, body temperature, sleep and activity can be stored on an electronic device after being collected by wearable sensors. Such data can be automatically uploaded to a cloud based server at pre-determined intervals or a user of an electronic device could use an app to manually upload the data to a cloud based server. Data uploaded to a cloud based server in this way can be transmitted from an electronic device by WIFI or mobile network, for example. It may desirable for certain data to be securely uploaded to a local server or cloud based server. In such circumstances, a wearable sensor and/or associated electronic device can be connected to the server by a medical professional to manually instruct transmission of data.

The multi-dimensional data in the memory is combined and manipulated by a processor to generate the enrichment indicator by comparing the multi-dimensional input data to a database of reference subjects with known clinical progression. The enrichment indicator can be compared to a pre-determined target indicator to identify patients whom are the best match for clinical trial or treatment or can be used to provide a prognosis on potential clinical development of a given subject. Results generated by the processor are displayed on an output such as a visual display unit or on paper.

Not all data collected on a patient is relevant to diagnosis of particular diseases. Embodiments of the invention are concerned with diagnosis and/or treatment of neurodegenerative diseases including Alzheimer's disease, vascular dementia, lewy body dementia, frontal temporal dementia, multiple sclerosis, Huntington's disease and Parkinson's disease, for example. Alzheimer's disease, vascular dementia and lewy body dementia can all present in the same way clinically, and are often confused with one another, and are all related members of a clinical category “dementia”. Each neurodegenerative disease has different criteria for diagnosis however there are common broad criteria that a medical professional will look for to determine whether the patient is suffering from a neurodegenerative disease of any kind.

Embodiments of the invention require that a first sub-set of data is collected on the patient which comprises two or more of: demographic information, medical history, clinical symptoms, subjective complaints, patient activity measured by wearable sensors and/or genetic risk factors. The first sub-set of data is typically collected by a general practitioner and stored in a first location.

A second sub-set of data is also collected which comprises two or more of: clinical tests, medical imaging, CSF analysis and/or blood based markers. The second sub-set of data may be collected by several medical professionals and stored in several different locations with no direct access between storage locations. The second sub-set of data may also include data collected by wearable sensors.

The first sub-set of data and the second sub-set of data are combined to form an enrichment indicator which has been shown to accurately identify patients suffering from the early stages of neurodegenerative disease by way of comparison of the enrichment indicator with a set of pre-determined target indicators. Comparison of the enrichment indicator with the pre-determined target indicators permits a medical professional to characterize a patient as either suitable or not suitable for treatment of a specific neurodegenerative disease. Suitable patients can then be selected accordingly for treatment. While the combination of measurements to generate an enrichment indicator can be done by applying a sequence of different thresholds all of which must be passed, a better performing enrichment indicator is usually obtained by modelling the interplay between different measurements through a supervised machine learning approach.

Table 1 (below) indicates a number of biomarkers which, when present within a defined range, are indicative of onset of a neurodegenerative condition.

In relation to dementia, the majority of sufferers are older than fifty five years of age. Analysis of patient images on MRI for medial temporal lobe structure volumes provides an indication as to risk of onset of dementia. Patients having a medial temporal lobe structure volume less than the fifth percentile of healthy age matched subjects are considered to be at high risk of developing dementia over a timeframe of several years. Patients having a medial temporal lobe structure volume less than the fifteenth percentile of healthy age matched subjects are considered to be at medium risk of developing dementia over a timeframe of several years. In conjunction with analysis of MRI scans, the patient will be assessed and scored in relation to cognition and clinical tests like the Clinical Dementia Rating (CDR) or the Mini Mental State Examination (MMSE). An MMSE score of less than twenty seven is considered to be indicative of a patient suffering from dementia. The patient will also be assigned a CDR; a CDR of between 0.5 and 1 is considered to be indicative of mild dementia symptoms, a CDR of between 1 and 2 is considered to be indicative of mild to moderate dementia symptoms and a CDR of 3 is considered to be indicative of severe dementia symptoms.

In relation to Alzheimer's Disease, several markers can indicate risk of onset of Alzheimer's Disease. A SUVR value of greater than 1.11 as measured by amyloid PET imaging with a given tracer, is considered to be abnormal as is a Aβ-42 value of less than 192 pg/mL, as measured from a cerebrospinal fluid (CSF) sample obtained through a lumbar puncture. Each of these abnormal values is considered to be indicative of a high risk of onset of Alzheimer's Disease. Blood based markers including a polypeptide value of greater than 2 and/or a apolipoproteint E (μg/ml) value of less than 1.75 are also considered to be indicative of a high risk of onset of Alzheimer's Disease. Genetic testing is also useful to determine if a patient is a carrier of ε4 or ε2 APOE carriers. ε4 carriers are considered to be indicative of a high risk of onset of Alzheimer's Disease whereas ε2 carriers are considered to be indicative of a low risk of onset of Alzheimer's Disease.

In relation to Vascular Dementia, analysis of patient images on MRI FLAIR for white matter affected by lesions provides an indication as to risk of onset of Vascular Dementia. A patient having a brain exhibiting greater than 20% of white matter affected by lesions is considered to be at high risk of onset of Vascular Dementia. In addition, a patient having suffered a stroke within three months of onset of dementia symptoms is considered likely to be suffering from Vascular Dementia.

In relation to Multiple Sclerosis, the majority of sufferers began exhibiting symptoms within the age range of twenty to fifty years. Analysis of MRI T2 FLAIR for lesions in at least two of four brain regions can indicate onset of Multiple Sclerosis. Patients having symptoms of Multiple Sclerosis will have their brain atrophy scanned from MRI T1 wherein atrophy of greater than 0.7%/year is considered abnormal. The patient's cerebrospinal fluid (CSF) IgG-albumin serum IgG-albinum ratio will also be measured wherein a ration of greater than 0.7 is considered abnormal. Abnormal brain atrophy and CSF IgG-albumin to serum IgG-albumin ratio values are considered to be indicative of onset of Multiple Sclerosis although not conclusive.

In relation to Huntington's Disease, analysis of striatum (caudate, putamen) atrophy and CAG repeats can indicate an increased risk of onset of Huntington's Disease. A progressive striatum (caudate putamen) atrophy of greater than 1%/year is considered to be an indicative of an increased risk of onset of Huntington's Disease. Genetic testing for CAG repeats highlights an increase in CAG repeats in relation to a particular patient. A healthy patient not suffering from Huntington's disease will exhibit between ten and thirty five repeats. This range is considered normal. A patient exhibiting between thirty six and thirty nine CAG repeats is considered to be at increased risk of onset of Huntington's Disease and a patient exhibiting in excess of forty CAG repeats is considered to be at high risk of onset of Huntington's Disease.

In relation to Parkinson's Disease, the majority of sufferers begin exhibiting symptoms after the age of sixty. However, some patients can exhibit symptoms of Parkinson's Disease at an earlier age if they also exhibit gene mutations to one or more of LRRK2, PARK2, PARK7, PINK1 and SCNA. Analysis of DAT scans for abnormal uptake ratio of putamen (PUR) and caudate (CUR) can indicate if a patient is suffering from Parkinson's Disease. An uptake ratio of PUR of less than 1.35 is considered abnormal and an uptake ration of CUR of less than 1.37 is considered abnormal.

TABLE 1

| Neurodegenerative | | |
|---|---|---|
| Condition | Biomarker Presence | Biomarker Range |
| Dementia | Medial temporal lobe atrophy | Medial temporal lobe structural |
| | | volumes <5th percentile of healthy |
| | | age matched subjects: high risk. |
| | | Medial temporal lobe structural |
| | | volumes <15th percentile of healthy |
| | | age matched subjects |
| Vascular dementia | White matter lesions on MRI | >20% of white matter affected by |
| | FLAIR | lesions |
| Alzheimer's disease | Amyloid accmulation on PET | Florbetapir F18 SUVR >1.11 |
| Alzheimer's disease | Decreased A-Beta values in CSF | Aβ-42 <192 pg/ml |
| Alzheimer's disease | Blood-based proteomics | Pancreatic olupeptide >2 (pg/ml) |
| | | Apolipoprotein E (μg/ml) |
| Alzheimer's disease | ApoE gene | Carriage of ϵ4 allele |
| Multiple sclerosis | White matter lesions on MRI | ≥1 lesion in two of four brain |
| | FLAIR | regions |
| Multiple sclerosis | Brain atrophy from MRI T1 | Atrophy >0.7%/year |
| Multiple sclerosis | Immunoglobulin G from CSF/ | Ratio CSF IgG-albunim ratio to |
| | serum | serum IgG-albunim ration >0.7 |
| Huntington's disease | Basal ganglia atrophy from | Striatum atrophy >1%/year |
| | MRI T1 | |
| Huntington's disease | Increased CAG repeats | >35 repeats: increase risk. >39 |
| | | repeats: high riskt |
| Parkinson's disease | Reduced dopamine on DAT | Uptake ratio in putamen <1.35 |
| | scans | Uptake ratio in caudate <1.37 |
| Parkinson's disease | Genetic analysis | Mutations in LRRK2, PARK2, |
| | | PARK7, PINK1, SNCA |

The second sub-set of data may include analysis of the predicted progression of a neurodegenerative disease and/or the predicted response of a neurodegenerative disease to a particular treatment. In particular, combination of predicted progression and/or response to treatment with basic patient data generates an enrichment indicator which can assist in identifying patient's whose disease is stable or who would not be expected to respond to treatment. Such patients can be excluded from selection criteria to improve the quality of the selection pool. The remaining selection pool should only include patients whose disease is predicted to progress and patients who are predicted to respond to a particular treatment. Alternatively, patients can be identified who would be expected to respond to a particular treatment and such patients can be removed from the selection pool as a shortlist of patients who might be susceptible to treatment.

The enrichment indicator may also be used to select treatments that are appropriate for a particular patient by comparing the enrichment indicator with a pre-determined target indicator.

During a clinical trial or during treatment, a patient's response to treatment can be monitored such that treatment can be varied if necessary in response to progression of a neurodegenerative disease or in the event that a patient responds to treatment of a neurodegenerative disease. For example, in a clinical trial some patients may be treated with a placebo and some patients may be treated with a medicament. Depending on an individual's response to treatment it may be desirable to change the medicament the patient is being treated with and in the case of a patient being treated with a placebo it may be desirable to swap the placebo for a medicament.

A particular enrichment tool according to embodiments of the combines patient demographics (age, gender), baseline cognitive assessments, biomarkers (which can include one or more of atrophy from structural MRI, amyloid burden from CSF A-Beta or Amyloid PET), and genotype (eg: carrying one or more copies of ApoE-4 allele). A more coherently progressing cohort can be identified thus improving clinical trial efficiency can be by enriching MCI populations using a combinatorial enrichment tool

Enrichment performance was measured using a hypothetical 2 year two arm study in aMCI patients to evaluate the impact of biomarker enrichment in two cohorts: 152 ADNI-I MCI subjects and 112 ADNI-II L-MCI subjects. This work evaluates how consistently typical clinical endpoints (MMSE, CDR-SB, ADAS-Cog) and related trial characteristics like effect size can vary on two cohorts recruited with the same criteria. It is then measured how these endpoints are influenced in absolute and relative terms by different enrichment strategies.

Hippocampal volume (HV) was extracted using the LEAP algorithm incorporated in the CE marked medical device, Assessa. Subjects were considered positive on hippocampal volume (HV+) with a hippocampal volume smaller than that of the 15th percentile of age matched healthy. Amyloid positivity (Am+) was defined from CSF through Abeta<192 and from PET imaging through AV45 SUVR>1.12. Finally, subjects were considered positive on ApoE-4 (E4+) if they carry at least one ε4 allele.

The combinatorial enrichment strategy provides a flexible means to identify patients that are positive in one or more of the described measurements, potentially selecting a patient population that is matched better to the treatment (e.g. more rapidly progressing population). This is evaluated with respect to two key trial characteristics: (i) the rate and homogeneity of clinical progression in the included trial cohort and (ii) the resulting screen failure rate. It is furthermore evaluated how different subject groups defined by their biomarker and genetic status compare regarding their MCI-AD conversion rate, change in clinical scores, amyloid levels, white matter lesion load, and ε4 status.

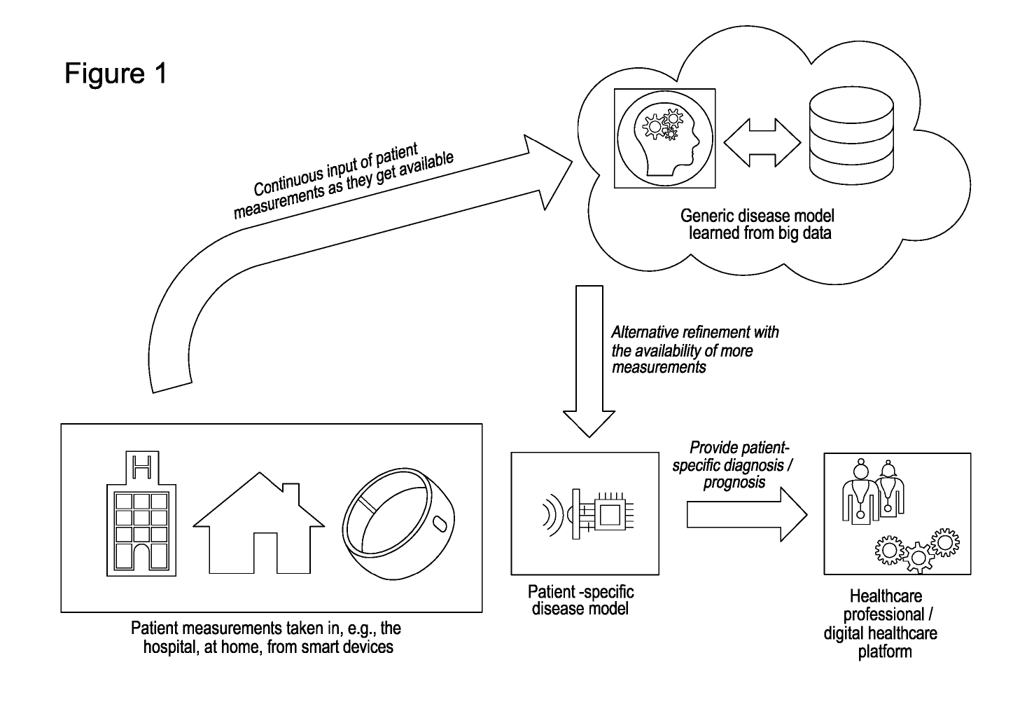

Rate of cognitive progression as measured by the different cognitive scores (MMSE, CDR-SB, ADAS-Cog) was evaluated for the unenriched group and the cohorts obtained when applying the enrichment criteria. Effect size was measured as mean change divided by the standard deviation of change in the different groups. Effect sizes in the two tested cohorts and with the three clinical endpoints are presented in FIG. 1 for the unenriched group as well as cohorts resulting from different enrichment strategies. All tested enrichment combinations of clinical scores and datasets resulted in substantial increases in the effect size. Substantial variability is observed between individual tests on individual datasets. For example, substantially higher effect sizes are measured with MMSE in ADNI II than in ADNI I, but CDR results shows much higher effect sizes in ADNI I than in ADNI II. Variability across the different endpoints in a single dataset goes down with more stringent enrichment, while the variability of a certain endpoints across datasets stays stable with enrichment.

All values are presented relative to the unenriched scenario and are averaged between the three clinical endpoints evaluated (MMSE, CDR-SB, ADAS-Cog).

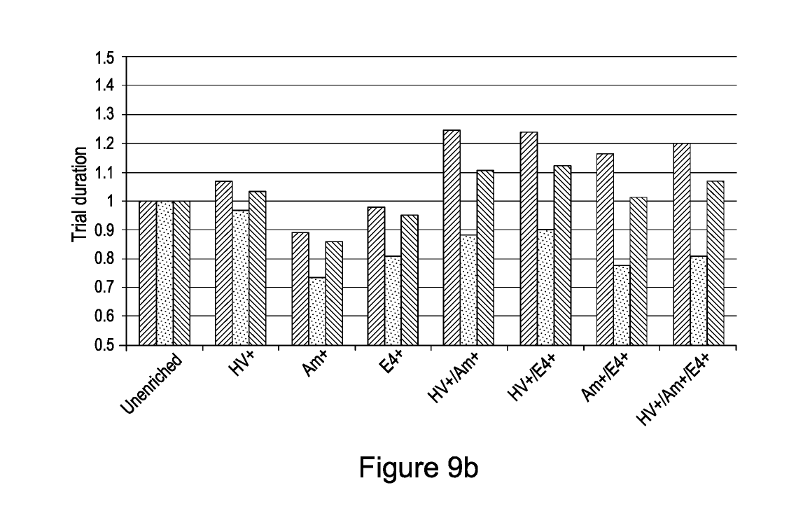

FIG. 9a shows: Screen failure rate (top left), number of subjects needed to screen (NNS) (top right), required sample sizes per arm (middle left) and trial cost (middle right) and trial duration (bottom left) with different enrichment strategies. All values are presented relative to the unenriched scenario and are averaged between the three clinical end points evaluated (MMSE, CDR-SB, ADAS-Cog). Base values for the combined cohort are NNS=2,222, Samplesize=771, trial cost=$71 Mio, duration=4.8 years.

A combinatorial enrichment tool provides flexibility in enrolling cohort with a desired effect size. This desired effect size may the maximum obtainable, or it can be designed to obtain a slower, coherently progressing sub-group. Enriching AD clinical trials with hippocampal volume can help to identify a rapidly and consistently progressing patient population and therefore improve trial design wrt required sample sizes, number of patients that need to undergo screening as well as trial cost. Applying HV enrichment in an Amyloid positive or ApoE-4 carrying group, the same effects are observed, providing even better improvements compared to an unenriched group. The described effects are observed across ADNI I and ADNI II populations, even though clinical progression is substantially different between both cohorts and between different clinical outcome measurements (CDR-SB vs MMSE vs ADAS-Cog). Tighter enrichment leads to less variability between the two patient populations (ADNI I/ADNI II). Combining both cohorts gives stable enrichment results with all measured clinical endpoints. Cut-points defined at the 40th and 25th percentile of healthy subjects vary less than 1% between both cohorts. Variability for the stringent cut point at the 10th percentile is 4.5%.

Combined enrichment outperforms enrichment with only one biomarker in all measured characteristics. Combined enrichment does not increase the number of subjects that need to undergo screening (reduced sample sizes vs increased screen failure rate).

Enriching with HV in the Amyloid positive group helps to screen out slow progressors. Progression rates after 1, 2 and 3 years are systematically around twice as high in the AM+/HV+ compared to the AM+/HV-group. Amyloid burden between AM+/HV+ and AM+/HV− is not significantly different suggesting that HV negativity adds additional information over a more stringent cut point on the Amyloid assay. Subjects that are AM−/HV+ (SNAPs) show significantly lower baseline MMSE than Am−/HV− subjects but a comparable rate of progression. They show significantly lower Amyloid load and significantly higher white matter lesion load. These points taken together suggest a potential baseline cognitive impairment (and structural degeneration) due to vascular dementia without the further progression observed in the subjects that are HV+ (which also have significantly higher percentage of MCI-to-AD converters).

All subjects in this met the following inclusion criteria: i) 1.5T (ADNI I) or 3T (ADNI II) MR scan available at baseline assessment; ii) Baseline assessment of Amyloid status (from CSF or Amyloid PET) available iii) Clinical scores (MMSE, CDR-SB, ADAS-Cog) available at baseline and at month 24

Subject characteristics are presented in the lower part of Table 2 (below).

TABLE 2
Subject characteristics. 8 subjects are CSF+/ PET−, 4 are PET+/CSF−.

| | ADNI I MCI | ADNI II MCI | Combined MCI |
|---|---|---|---|
| N (female %) | 152 (34%) | 107 (48%) | 259 (39%) |
| Age | 74.7 ± 7.5 | 71.2 ± 7.7 | 73.2 ± 7.8 |
| % Amyloid | 76% | 70% (CSF)*, 66% | 74% (all CSF)*, 72% |
| positive | (CSF) | (PET)* | (CSF/PET)* |
| % ApoE 4 carriers | 53% | 54% | 53% |
| MMSE BL/M24 | 26.9 ± 1.8/25.3 ± 3.9 | 27.7 ± 1.8/25.8 ± 3.4 | 27.2 ± 1.9/25.5 ± 3.7 |
| CDR-SOB | 1.51 ± 0.84/2.98 ± | 1.79 ± 1.06/2.78 ± | 1.62 ± 0.94/2.90 ± |
| BL/M24 | 2.13 | 2.23 | 2.17 |
| ADAS-Cog | 18.04 ± 6.78/22.42 ± | 18.66 ± 6.87/21.84 ± | 18.30 ± 6.81/22.18 ± |
| BL/M24 | 9.73 | 10.68 | 10.12 |
| % MCI-AD conv. | 18/34/42 | 15/33/44 | 17/34/43 |
| (1 y/2 y/3 y) | | | |
| HV | 1632 ± 301 | 1721 ± 328 | 1669 ± 315 |
| | ADNI I CN | ADNI II CN | Combined CN |
| N (female %) | 222 (48%) | 222 (53%) | 444 (50%) |
| Age | 76.3 ± 5.1 | 72.8 ± 6.0 | 74.5 ± 5.8 |
| MMSE BL | 29.0 ± 1.0 | 29.0 ± 1.3 | 29.0 ± 1.2 |
| CDR-SOB BL | 0.00 ± 0.09 | 0.02 ± 0.13 | 0.01 ± 0.11 |
| ADAS-Cog BL | | | |
| HV | 1956 ± 254 | 1953 ± 243 | 1954 ± 246 |

Table 3 (below) gives an overview on the percentile cut points in the two cohorts as well as the combined group. Stable cut points are observed across the cohorts, suggesting generalizability of the described methods.

| | 40th | 25th | 10th |
|---|---|---|---|
| | percentile | percentile | percentile |
| ADNI I | 1896.6 mm³ | 1823.8 mm³ | 1571.9 mm³ |
| ADNI II | 1907.8 mm³ | 1835.7 mm³ | 1644.7 mm³ |
| Combined | 1905.4 mm³ | 1834.8 mm³ | 1611.6 mm³ |

Included are the 321 (late) MCI and 179 mild AD subjects from the ADNI I/II cohorts for which an Amyloid marker is available from PET or CSF. Clinical assessment at baseline was required with CDR-SB/ADAS-Cog/FAQ and a follow-up on ADAS-Cog was also required after 12 months. Evaluated exemplarily were the impact individual/combinatorial biomarkers can have on the identification of subjects rapidly progressing on ADAS-Cog as measured through the effect size (mean change divided by standard deviation). Inclusion criteria could be defined around other patient characteristics that may be desirable for a particular trial. We assessed the impact an application of the following markers (cutpoints) can have when applied sequentially or in a combinatorial machine learning model: (1) FAQ (included with FAQ>0), (2) CDR-SB (included with CDR-SB>0.5), (3) amyloid positivity (cutpoints on PiB PET/CSF A-beta as widely used), (4) hippocampal volume, HCV (continuous cutpoints validated). Automated hippocampal volumetry and statistical modeling was performed in a research environment of the CE marked medical device Assessa.

Where multiple measurements are combined, results are presented for a Sequential approach with the cut points defined above as well as a supervised Machine learning approach. A linear regression model to predict change in Adas-Cog is employed in the supervised approach. In 1,000 runs, models are trained on 80% of data and tested on the remaining data. Results are averaged over all independent runs. Cut points on continuous variables (HCV in the sequential approach, output of supervised method) are defined in a way to arrive at the predefined screening failure rates listed in Table 4 below.

The presented results show different approaches for selection of suitable patients into a trial based on multiple measurements. The results show how by modeling the interplay between different measurements through a supervised machine learning approach, an improved prediction of subjects at higher risk of deteriorating on a relevant endpoint can be achieved. Especially continuous clinical scores like FAQ and CDR-SB can be integrated much more efficiently in a statistical modeling approach compared to a sequential approach which leads to significant screen failure rates even at the conservative cut points employed here. Statistical modeling also provides a natural means of integrating demographic information like age or education. Combining the relatively easily available measurements of CDR-SB, FAQ and HCV yields the same performance improvement as the significantly more expensive and invasive combination of Amyloid status and HCV. More work will be done to validate more advanced machine learning techniques, other relevant biomarker and patient-demographic data as well as the impact on other trial characteristics like other endpoints and trial cost and time.

A generic disease model as shown in FIG. 1 and envisaged by the present invention is based on a large dataset of measurements and information taken from multiple patients over a significant time period. Such measurements and information are anonymized and uploaded periodically to a generic, data disease model. Generic disease models are required for each individual disease as each disease displays unique symptoms and requires different measurements and information to be taken from patients in order to monitor development of the disease.

The present invention has been postulated as suitable for chronic diseases including: the diseases causing dementia (eg: alzheimers), mental health disorders, HIV/AIDS, cancer, asthma, diabetes, for example, but is not limited in use to such diseases. The present invention is suitable for use in any disease which requires ongoing monitoring and which has displayed itself in a sufficient number of patients.

A dynamic disease model according to embodiments of the present invention is derived from a generic disease model and is unique to a particular patient. Initial patient data is used to select data in the generic disease model which closely corresponds to the patient. From this, an initial prognosis and/or treatment plan is generated based on the content of the dynamic disease model.

The dynamic disease model is updatable with additional patient measurements and/or information in order to refine the initial prognosis and/or treatment plan as the patient's disease progresses or more patient data gets available.

Taking a disease model for asthma as an example, such patient data and/or measurements might include: peak flow rates, preventative inhaler dosage, preventative inhaler treatment plan, preventative inhaler actual frequency of use, reliever inhaler dose, reliever inhaler frequency of use, patients perception of how their asthma is controlled, any attacks or episodes of shortness of breath, trigger factors, weight, age, height, demographic, for example.

Taking a disease model for dementia as a further example, such patient data and/or information might include: demographic information about the individual (gender, age, educational history . . . ), the output of cognitive tests, the output of a depression score, blood tests to assess vitamin deficiencies, the patient's genotype such as their APOE4 carrier status, vascular risk factors, measurements from brain scans, measurements from CSF assays, results from blood tests of dementia risk-factors.

Upon diagnosis of a disease or condition, a medical professional can generate the dynamic disease model. The dynamic disease model is stored on secure digital storage medium and can be updated either instantaneously when further patient measurements and/or information are available or on demand. Upon each update, the dynamic disease model uses an algorithm to refer to the generic disease model and generate an updated prognosis and/or suggested treatment plan. A medical professional can thus identify how patient's with similar symptoms and measurements were treated, how effective such treatment was and how other patient's disease or condition progressed in comparison to the actual patient being assessed. The disease model can also be used to extract useful information for patient treatment from these high-dimensional datasets.

Patient measurements and/or information can be obtained in any number of ways. In the simplest form of the present invention, patient measurements and/or information may be obtained using suitable techniques and written down on paper. The patient measurements and/or information can then be uploaded manually to the patient's dynamic disease model by an operator.

Alternatively, a tablet computer could be used by a medical professional or carer to enter patient data and/or information. A tablet computer can also be used by the patient to answer simple questions relating to his or her condition, their feelings or their activities, for example.

In a particular embodiment of the present invention, the dynamic disease model is stored on a smart device such as smart watch or pendant or device mounted to the wall(s) of the patient's home. The dynamic disease model can be uploaded onto the smart device by either wired communication such as USB or wireless communication such as WiFi or Bluetooth. Alternatively, the dynamic disease model can be stored on removable storage media compatible with the smart device.

The smart device allows for automated monitoring of certain patient measurements and/or information. For example, a smart device used for monitoring a patient suffering from dementia can monitor a patient's location to identify whether the patient could remember how to get to and from a particular place within the home or in the wider community. Such a feature for a smart device carried by the patient would also enable emergency services to locate the patient should the patient get into difficulty or get lost.

The smart device can also monitor the patient, such as vital signs including heart rate and respiration, facial expressions, movement and or gait, to identify if the patient is in distress. Upon determining that a patient is in distress the smart device can communicate with the patient or people involved in the looking after the patient to enquire whether the patient requires assistance. The patient or others contacted would then have the option to contact emergency services for assistance. Depending on parameters set by a medical professional and data sent by the smart device to emergency services, an ambulance can be sent if required or a first responder or nurse if the patient's condition is not serious. In this way, appropriate emergency intervention can be provided, but inappropriate hospitalization avoided.

Some embodiments of smart devices can have input means such as a touch screen or buttons that the patient, a medical professional or a carer can use to input patient measurements and/or information. Such an embodiment also enables a patient to answer simple questions about how he or she feels, or whether planned treatment and/or care has been delivered, and this information can be used to refine the dynamic disease model.

When the patient visits a medical professional, the smart device can communicate with a computer or tablet to display the dynamic disease model. The smart device can function in one of two ways: 1) it can update the dynamic disease model as and when new patient data and/or information is captured; or 2) it can store new patient data and/or information and update the dynamic disease model only when it is in communication with a specified computer or tablet.

In other embodiments, the smart device can store an adaptive or dynamic treatment plan in addition to or in place of the dynamic disease model. The treatment plan sets out the treatment prescribed for the patient and is updatable based on patient measurements and/or information which can be captured by the wearable device, or otherwise.

The treatment and/or care plan can be updated remotely in response to patient data and/or information which is captured by the wearable device. In a particular embodiment of the invention, patient data and/or information is captured by the smart device and then communicated instantaneously by the Global System for Mobile Communications (GSM) to a remote processing device. The remote processing device receives the patient data and/or information and relays the patient data and/or information to a server containing a global disease model.

The global disease model recognizes the patient data and/or information and activates an algorithm to compare the patient data and/or information to data and/or information stored in the generic disease model. Based on the comparison step, an output is sent to the dynamic disease model stored on the wearable device by GSM to update the dynamic or adaptive disease model to take into consideration the patient data and/or information captured by the wearable device.

The dynamic disease model produces an output to update the treatment plan with updated treatment for the patient.

The treatment plan can be set with an alarm in the case of the patient exhibiting behavior or symptoms which indicate that the patient's health is at risk. As an example, a Global Positioning Satellite (GPS) enabled smart device that is carried by the patient (wearable) can monitor a patient's location. In the event that GPS indicates the patient is lost, i.e. by detecting that the patient has been walking around in a circle for a certain amount of time, the treatment plan can request input from the patient via the wearable device to indicate whether the patient needs assistance. In the event of an affirmative or no response, the wearable device can send a signal to a monitoring centre indicating that the patient should be contacted and if the patient is uncontactable, emergency services can be notified.

A smart device that is not wearable may monitor the patients' position within their home or care-home to record their amount and type of activity, eg: whether a dementia patient is getting out of bed, moving to other parts of the house etc and this can help assess whether that patient is functioning independently.

The treatment and/or care plan can also be used as a tool for supporting development and evaluation of new drug and non-drug treatments. In such a scenario, the smart device would capture patient data and/or information and send this to a remote processing device by GSM or WiFi or similar means. The remote processing device would relay the data and/or information to either a person involved in the care of that patient, or a big data repository of clinical trial data. The patient data and/or information can be reviewed manually by the person involved in that person's treatment and care or autonomously by an algorithm in order to monitor the effect of the drug or non-drug treatment on the patient. Based on how the patient is responding to the drug or non-drug treatment, the treatment plan can be updated to vary the treatment, terminate the treatment or maintain the treatment. In the event that the wearable device captures data and/or information that indicates the patient is suffering from unforeseen or undesirable side effects from the drug of non-drug treatment, that information can be captured as part of assessing the safety of that experimental treatment, but also the treatment plan can be updated with an alarm code indicating to the patient that they require medical attention. Alternatively, the treatment plan can be pre-conditioned with an alarm which would indicate to the patient that they require medical attention if certain parameters become evident in the patient.

### EXAMPLES

This example demonstrates an application of the invention in patients with cognitive difficulties including MCI and dementia.

Identification of patients with cognitive impairment (eg: MCI, dementia) at the highest risk of future cognitive or functional decline is important clinically. Much work has been done on the predictive power of clinical tests alone. Those older adults that show less cognitive decline as they age are less likely to report comorbid medical conditions and decreases in activities of daily living than people who exhibit more decline. Intervening earlier in the disease process can make treatments more effective and allow better management of that patient's care. It has also been suggested that people who decline cognitively and functionally, more quickly than others have a poorer prognosis and require more resources to manage their care. Brain biomarkers are significant contributors in the diagnosis of dementia. In recent years research has shown that predictive models of dementia risk are more accurate when they include multiple risk factors.

There are already several well characterized biomarkers for dementia (CSF, PET, structural MRI imaging that can be used to support diagnosis, all of which have been qualified as biomarkers to enrich clinical trials by EMA). Recent literature shows the advantage of combining them and integrating them with existing diagnostic tests like neuropsychological testing. Research has highlighted the benefit of combining MRI measurements with cognitive testing when trying to predict dementia risk. Specifically studies have shown that combining cognitive tests and regional brain volume measures were the best at predicting conversion to dementia in patients with mild cognitive impairment.

An embodiment of this invention, provides a prognosis index that predicts cognitive and functional decline from structural imaging and baseline clinical assessment. This index works by generating a patient specific disease model that predicts cognitive and functional decline, by comparing multiple measurements obtained from the patients with a generic disease model incorporating a large database of normal and demented patients who have been followed up for at least 24 months. The prognostic index is an output of this personalized model, and a practical output of this model is patients being classified as Rapid Cognitive Decliners (RCD) or Rapid Functional Decliners (RFD) if the rate of decline is predicted to be >=8 MMSE points or >=10 FAQ points over 2 years respectively. The prognosis index is based on the medial temporal lobe atrophy index, MTAI (combination of structural volumes for the hippocampus, amygdala, temporal horn and lateral ventricles) as extracted with a hospital based device from the baseline MR image as well as the relevant demographics, baseline clinical score, MMSE or FAQ. Both features are used for Gaussian mixture population estimation. The difference between rapidly progressing and non-progressing (cognitively or functionally) populations was modelled as a sigmoid function in order to obtain a likelihood to belong to the progressing or stable subject groups. Class-likelihood indices derived from both MTAI-MMSE and MTAI-FAQ were used to predict RCD and RFD subjects on a test dataset. Prediction accuracies of 74% and 80% were respectively obtained when using both sources of information. This compares to 68% (MTAI only) and 68% (MMSE only) for cognitive decline. Accuracies of 70% (MTAI only) and 73% (FAQ only) where achieved with individual measurements to predict functional decline. These results show how the defined disease model of structural imaging and baseline clinical assessment (MMSE/FAQ) gives an improved prognosis as to whether a subject is likely to rapidly deteriorate in cognition (RCD) or function (RFD) compared to an individual assessment of the clinical score or structural imaging.

The patient specific model is dynamic, in that it can be updated with additional measurements collected later, including repeat brain scans, or additional patient measurements, including biomarkers of amyloid, or genotype information (such as APOE4 carrier status).

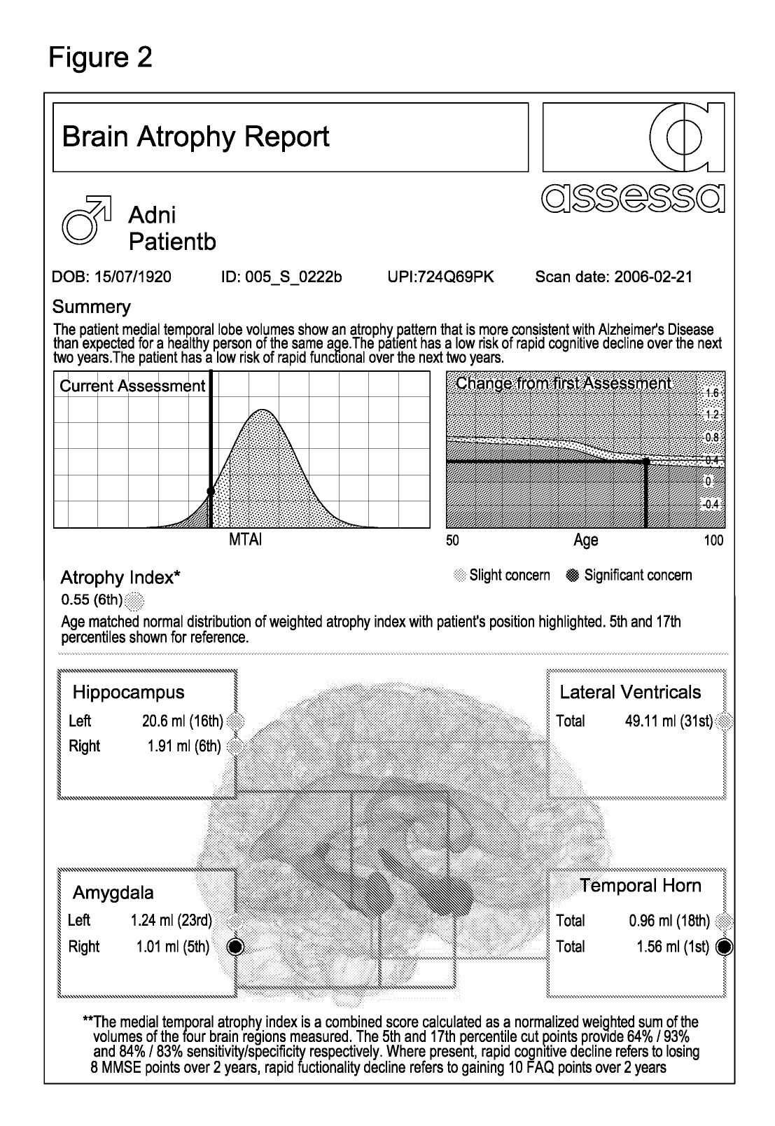

An example for the refinement of a disease model is the addition of longitudinal structural information to the MTAI index referred to above. With the availability of only baseline structural information, a relatively simple, patient-specific instance of the global disease model is used (ie the baseline MTAI (combining multiple structural volumes at baseline)). This relatively basic model provides an accuracy of 83% for the discrimination between healthy and dementia subjects, only improving from 79% and 78% when using hippocampal or amygdala volume alone respectively without disease modelling. With the availability of longitudinal structural information, a refined instance of the model is available for a specific patient. On the used test dataset, this additional information can improve classification accuracy to 89%. Combining structural information with non-imaging data as exemplarily shown above can further improve prognostic/diagnostic accuracy, showing how an the fit of patient data to a reference model learned from a large dataset can iteratively be refined. See FIG. 2.

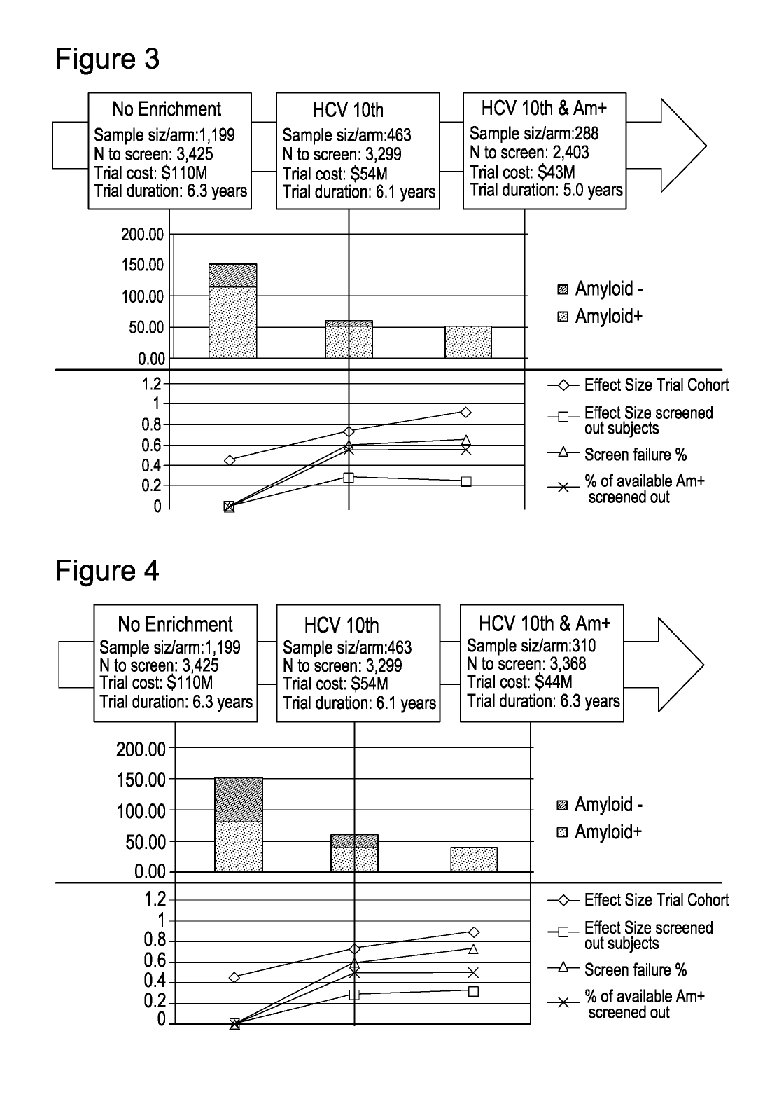

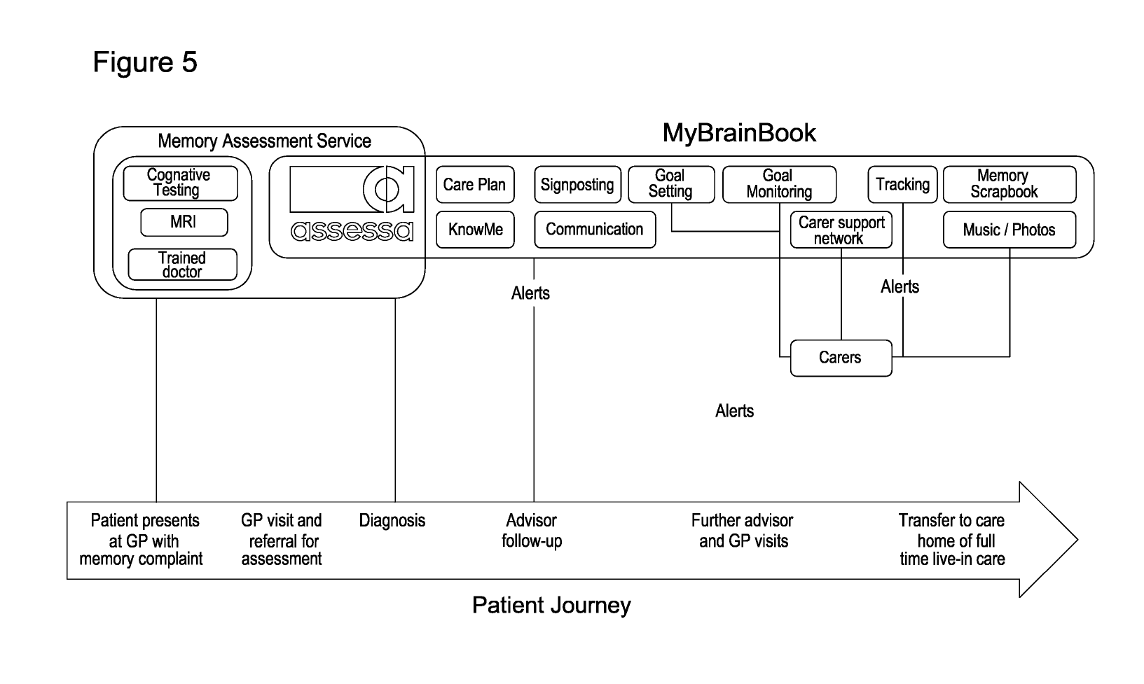

A second example of the dynamic nature of the disease model is illustrated in the FIGS. 3 and 4 through the combination of structural imaging with an amyloid marker or genotype: in this example it is shown that the rate of cognitive decline is higher with multiple positive markers (structural imaging & amyloid and structural imaging & genotype). In this particular application of the invention, the prognostic information can be used to enrich clinical trials of new treatments.

A further example for the dynamic nature of the disease model is in the integration of post-diagnosis information: Post-diagnosis information is collected both from subsequent hospital visits (eg: repeat brain scans or cognitive assessments) and from other data and information entered by the patient and those that are involved in their well-being, both in their family and outside. The availability of such information can update the patient-specific instance of the disease model. The device or devices for collecting this post-diagnostic information is located in the patient's home. It includes a profile of the patient populated by the patient and their family to assist those caring for them to provide more personalized care and also to make the device “sticky” so the patient and their immediate family want to continue to engage with it: content like music, pictures, poems that the patient enjoys, information about their preferences as they may change. The home-based system according to the invention uses the information about the patient (demographics, information from diagnosis and subsequent assessment) to update the patient-specific dynamic model (which may have been an output of hospital-based system according to the invention or may have been provided by an entering data separately) and record the patients own goals and priorities. The output of the system includes a dynamic care plan, and also the ability to input data manually or automatically from smart devices that both monitor whether the patient has been meeting their personal objectives, and whether the patient's dynamic disease model of cognitive and functional decline is predicting change in what they can achieve accurately, and if not, to update this

For example, if the patient has goals to go out to the day centre and walk around the park twice per week, whether this is achieved can be tracked.

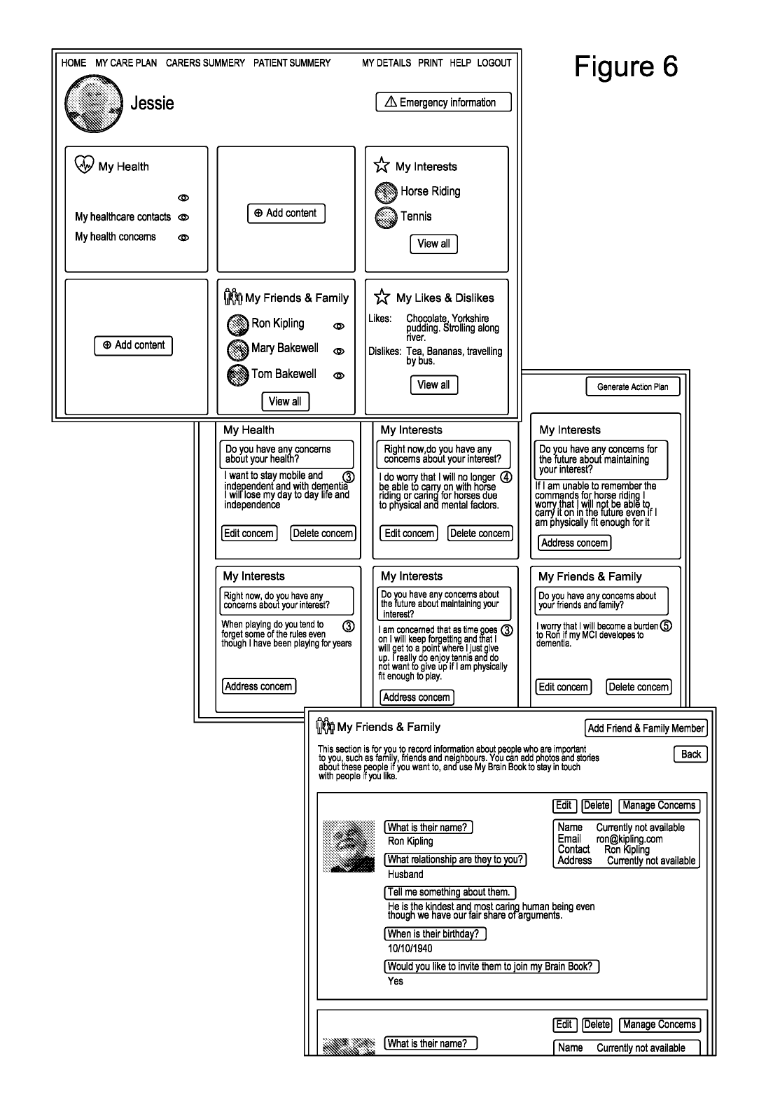

A feature of the hone-based system is that it can message the emergency services with information about the patient that can inform how any calls to these services are dealt with, and avoid inappropriate responses. The schematic in FIG. 5 shows how the hospital based system and home based system of the invention, interact in the patient journey.

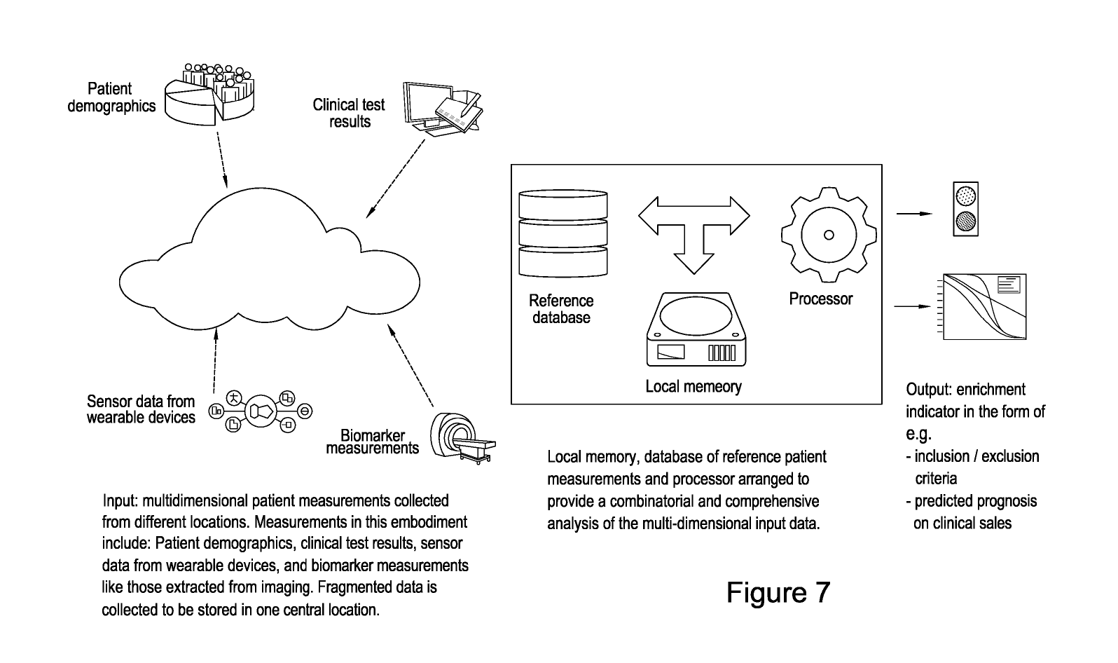

Information, and care action plan. This patient-facing-interface can collect further information and data about the patient to update the dynamic personalized disease model, outputs from which include care and treatment plans and emergency plans that can be shared with emergency services. See FIG. 6.

1. A system for selecting a patient for treatment, the system comprising:
i) a memory for storing data relating to patients, wherein the data includes a plurality of patient specific data types from a first data set and a plurality of patient specific data types from a second data set, different to the first data set;
ii) a first data input source for inputting patient specific data into the memory from the first data set and a second data input source for inputting patient specific data into the memory from the second data set;
iii) a processor for manipulating and/or combining patient specific data stored in the memory from the first data set and data stored in the memory from the second data set to define an enrichment indicator and comparing said enrichment indicator to a pre-determined target indicator, wherein, each patient's enrichment indicator defines a unique variable and the pre-determined target indicator applies a threshold to the enrichment indicator such that patients whose unique variable corresponds to the pre-determined target indicator are selected; and
iv) an output for displaying selected patients.

2. The system for selecting a patient for treatment according to claim 1, wherein the pre-determined target indicator is identified from a pool of at least 500 data points.

3. The system for selecting a patient for treatment according to claim 1, wherein the processor is configured to apply a machine learning algorithm to combine different combinations of the plurality of data input sources to identify a refined selection criteria for selecting patients for clinical trial or treatment of a neurodegenerative disease.

4. The system for selecting a patient for treatment according to claim 1, wherein the machine learning algorithm is a linear regression algorithm.

5. The system for selecting a patient for treatment according to claim 1, wherein the memory is accessible by a plurality of network or internet connected devices.

6. The system for selecting a patient for treatment according to claim 1, wherein the memory is cloud based.

7. The system for selecting a patient for treatment according to claim 1, wherein the first data set comprises at least two data types selected from i) demographic information; ii) medical history; iii) clinical symptoms; iv) subjective complaints and v) activity from wearable sensors.

8. The system for selecting a patient for treatment according to claim 1, wherein the second data set comprises at least two data types selected from vi) clinical test results; vii) imaging; viii) CSF analysis; ix) blood based markers, x) genetic risk factors, xi) predicted neurodegenerative disease progression, xii); and xiii) and v) activity from a wearable sensor.

9. The system for selecting a patient for treatment according to claim 1 wherein the enrichment indicator is defined by combining two or more data types from the first data set with two or more data types from the second data set.

10. The system for selecting a patient for treatment according to claim 1, wherein the first data input source and/or the second data input source is a wearable device or portable electronic device.

11. A method of selecting a patient for treatment, the method comprising the steps of:
(a) collecting a first sub-set of patient specific data comprising at least two of: i) demographic information; ii) medical history; iii) clinical symptoms; iv) subjective complaints and v) activity from a wearable sensor;
(b) collecting a second sub-set of patient specific data comprising at least two of: vi) clinical test results; vii) imaging; viii) CSF analysis; ix) blood based markers and x) genetic risk factors;
(c) combining the first sub-set of patient specific data and the second sub-set of patient specific data to define an enrichment indicator;
(d) comparing the enrichment indicator with a set of pre-determined target indicators;
(e) characterizing one or more patients from which the first and second sub-sets of patient specific data were derived as being suitable or non-suitable for treatment of a neurodegenerative disease in accordance with step (d); and
(f) selecting one or more patients for treatment.

12. The method of selecting a patient for treatment according to claim 11, wherein the neurodegenerative disease is Alzheimer's disease.

13. The method of selecting a patient for treatment according to claim 11, wherein the neurodegenerative disease is dementia.

14. The method of selecting a patient for treatment according to claim 11, wherein the neurodegenerative disease is vascular dementia.

15. The method of selecting a patient for treatment according to claim 11, wherein the neurodegenerative disease is multiple sclerosis.

16. The method of selecting a patient for treatment according to claim 11, wherein the neurodegenerative disease is Huntington's disease.

17. The method of selecting a patient for treatment according to claim 11, wherein the neurodegenerative disease is Parkinson's disease.

18. The method of selecting a patient for treatment according to claim 12, wherein the first sub-set of patient specific data specifically comprises increased impairment on episodic memory.

19. The method of selecting a patient for treatment according to claim 10, wherein the second sub-set of patient specific data specifically comprises one or more of: amyloid accumulation on PET; decreased A-Beta values in CSF; the markers in table 1 or 2 and ApoE status.

20. The method of selecting a patient for treatment according to claim 10, wherein patients below fifty five years of age are removed from the enriched dataset.

21-42. (canceled)
